(19) United States
(12) Patent Application Publication
(10) Pub. No.: US 2006/0222718 A1
(43) Pub. Date: Oct. 5, 2006

(54) Aqueous pharmaceutical solution comprising oxymetazoline and/or xylometazoline

(76) Inventors: Frank Böhme, beethovernring (DE); Bernd Eschenbach, Hattersheim (DE); Dagmar Färber, Heppenheim (DE); Claudia Hey, Brucknerweg (DE); Barbara Pfaff, Schöllkrippen (DE); Marion Tschaikin, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/567,845

(22) PCT Filed: Jul. 14, 2004
(86) PCT No.: PCT/EP04/07780

§ 371 Date: Feb. 10, 2006

(30) Foreign Application Priority Data
Aug. 13, 2003 (DE) 10337186.9

(51) Int. Cl. A61K 31/4164; A61K 33/32

(52) U.S. Cl. 424/641; 514/396

(57) ABSTRACT

The present invention relates to a stable aqueous solution comprising oxymetazoline and/or xylometazoline, a zinc salt and a buffer salt. The aqueous solution is particularly suitable for local administration into the nose for decongesting the mucous membrane.

The present invention relates to a stable aqueous solution comprising oxymetazoline and/or xylometazoline, a zinc salt and a buffer salt. The aqueous solution is particularly suitable for local administration into the nose for decongesting the mucous membrane.

Oxymetazoline [6-tert-butyl-3-(4,5-dihydro-1-H-imidazol-2-ylmethyl)-2,4-dimethylphenol] and xylometazoline [2-(4-tert-butyl-2,6-dimethylbenzyl)-4,5-dihydro-1-H-imidazole] are imidazole α-sympathomimetics with a vasocon-strictive action which are preferably employed locally for decongesting the mucous membrane in the nose. In particular, aqueous solutions are used here.

Oxymetazoline and xylometazoline are unstable in aqueous solution. Although oxymetazoline and xylometazoline are more stable in aqueous solution in the form of their hydrochloride salts, undesired hydrolytic degradation of the active compounds, in particular due to hydrolytic cleavage of the imidazole ring, does also occur in these on storage, in particular at elevated temperature. Undesired degradation products, which may be associated with the risk of harmful side effects on use of the aqueous solution as medicament, form and the content of active compound drops. Overall, the shelf life of the aqueous solution is reduced.

As a consequence of hydrolytic cleavage of the imidazoline ring, oxymetazoline and xylometazoline in aqueous solution form, in particular, N-(2-aminoethyl)-2-[4-(1,1-dimethylethyl)-3-hydroxy-2,6-dimethylphenyl]-acetamide and N-(2-aminoethyl)-2-[4-(1,1-dimethylethyl)-2,6-dimethylphenyl]acetamide respectively. These degradation products are also listed as impurities in the monographs relating to these active compounds in the European Pharmacopoeia 2003.

Hydrolytic degradation of oxymetazoline and xylometazoline can be prevented if a non-aqueous solvent, for example oils or organic solvents, are employed instead of the aqueous solvent. However, oils have a comparatively higher viscosity and poorer ability to wet hydrophilic surfaces, which stands in the way of fine distribution of the active compound present on the nasal mucous membrane in the case of nasal administration. Organic solvents are usually not toxicologically acceptable and/or result in irritation of the nasal mucous membrane. Non-aqueous formulations also appear less suitable for the development of active-compound solutions owing to their viscosity or possible interactions with packaging materials and dispensing systems, in particular those made from plastics. This applies in particular to spray bottles made from plastics, which are widely used for rhinological agents.

The object of the present invention was to provide an aqueous solution of oxymetazoline and/or xylometazoline having increased stability. In particular, the aim was to reduce hydrolytic degradation as a consequence of cleavage of the imidazoline ring.

Surprisingly, it has been found that a stable aqueous solution comprising oxymetazoline and/or xylometazoline can be obtained if the latter comprises a zinc salt and a buffer salt in addition to the oxymetazoline and/or xylometazoline. The present invention therefore relates to an aqueous solution comprising at least oxymetazoline and/or xylometazoline, a zinc salt and a buffer salt.

For the purposes of the invention, an aqueous solution is present if at least some of the solvent present consists of water. Further solvent constituents that may be present are all solvents which are suitable for nasal administration, in particular alcohols, such as, for example, ethanol, propanol, propanediol or glycerol. The aqueous solution preferably comprises water or ethanol/water mixtures as solvent, the solvent particularly preferably consists of water.

In the aqueous solution according to the invention, oxymetazoline and/or xylometazoline is preferably present in the form of one of its pharmaceutically tolerated salts, such as, for example, as hydrochloride or as nitrate. Oxymetazoline and/or xylometazoline are particularly preferably each present as hydrochloride. Any oxymetazoline or xylometazoline amount data contained in the present patent application in each case relate to the corresponding hydrochloride salts. Other salt forms of oxymetazoline nitrate or xylometazoline nitrate are employed in an equimolar amount corresponding to the respective hydrochloride salt.

As zinc salt, use can be made in accordance with the invention of all pharmaceutically acceptable zinc salts. Preference is given to zinc chloride, zinc lactate, zinc sulfate, zinc citrate, zinc acetate, zinc histidinate, zinc orotate, zinc aspartate and/or zinc gluconate. The zinc salt present is particularly preferably zinc gluconate.

For the purposes of the present invention, buffer salts are the salts of weak acids with strong bases or of weak bases with strong acids, which dissociate completely in aqueous solution and form a buffer system in the presence of the respective salt-forming acid or base. Buffer salts which can be used in accordance with the invention are, for example, alkali metal salts, in particular the sodium and/or potassium salts, of pharmaceutically usable weak organic or inorganic acids, such as, for example, acetic acid or citric acid, or boric acid. The buffer salt present is particularly preferably sodium citrate.

According to an advantageous embodiment of the invention, the aqueous solution furthermore comprises a pharmaceutically acceptable acid or base. The acid or base present can be any pharmaceutically acceptable acids or bases which do not result in incompatibilities with the other constituents of the aqueous solution according to the invention. The acid present is preferably the respective acid forming the buffer salt, i.e. the acid form of the anion present in the buffer salt, or the base present is preferably the base forming the buffer salt, i.e. the base form of the cation present in the buffer salt. For example, the aqueous solution according to the invention may, besides the buffer salt sodium citrate, comprise citric acid, i.e. the acid form of the citrate ions present as anion in the buffer salt, or NaOH, i.e. the base form of the sodium ions present as cation in the buffer salt.

The aqueous solution according to the invention can, for nasal administration, be applied in all medicament forms which are suitable for nasal administration, such as, for example, nasal drops or nasal sprays, by means of dispensing devices suitable for this purpose, such as bottles with drop device or nasal spray pumps.

According to an advantageous embodiment of the invention, the aqueous solution has a pH of pH 4 to pH 7.5, preferably a pH of pH 5.0 to pH 7.2, particularly preferably a pH of about pH 6.0. The pH can be set accurately here by addition of the acid or base corresponding to the buffer salt and/or by addition of another physiologically tolerated acid or base. For example, the desired pH of a sodium citrate-containing solution can be set by addition of the acid form of the anion present in the buffer salt, i.e. by addition of citric acid, or by addition of the base form of the cation present in the buffer salt, i.e. by addition of NaOH, and/or by addition of another acid or base, in particular by addition of hydrochloric acid or sodium hydroxide solution.

In order to improve the ability of the aqueous solution to be tolerated on administration to the nasal mucous membrane, it is advantageous to formulate it as isotonic solution. Isotonicity is present at an osmolality of about 280 mOsm. The osmolality can be set by variation of the amounts of the dissolved substances present in the aqueous solution besides oxymetazoline and/or xylometazoline, i.e. the zinc salt, buffer salt and any further substances present, and/or by addition of an isotonicity agent, preferably a physiologically tolerated salt, such as, for example, sodium chloride or potassium chloride, or a physiologically tolerated polyol, such as, for example, a sugar alcohol, in particular sorbitol or glycerol, in the concentration necessary for rendering isotonic. The osmolality is preferably set by selection of the dissolved substance amounts present anyway in the aqueous solution, so that it is not necessary to add an isotonicity agent.

The aqueous solution according to the invention comprises oxymetazoline and/or xylometazoline in a concentration of 0.005% by weight to 1.0% by weight. Oxymetazoline and/or xylometazoline is preferably present in a concentration of 0.01% by weight to 0.5% by weight, very particularly preferably in a concentration of between 0.05% by weight to 0.1% by weight.

The aqueous solution according to the invention comprises that the zinc salt in a proportion of 0.1% by weight to 10% by weight. The zinc salt is preferably present in a proportion of 1.8-6.0% by weight. The buffer salt is present in a proportion of 0.01% by weight to 3.0% by weight. The buffer salt is preferably present in a proportion of 0.2-1.5% by weight.

According to an advantageous embodiment, the aqueous solution according to the invention comprises 0.005% by weight to 0.1% by weight of oxymetazoline hydrochloride or xylometazoline hydrochloride, 1% by weight to 10% by weight of zinc gluconate and 0.5% by weight to 5% by weight of citrate and has a pH of about 6.

According to an advantageous embodiment of the invention, the aqueous solution comprises only one of the active compounds oxymetazoline and xylometazoline in the form of its hydrochloride salt.

The examples explain the invention without being restricted thereto.

### EXAMPLE 1

| Oxymetazoline hydrochloride | 2.625 | g |
|---|---|---|
| Zinc gluconate | 300.00 | g |
| NaOH, 1 mol/litre | 51.20 | ml |
| Sodium citrate | 41.440 | g |
| Water for injection purposes | 4604.735 | g |
| pH | 5.99 | |
| Osmolality | 280 | mOsm/l |

Preparation Process

Water for injection purposes is initially introduced. The weighed-out substances are added in portions with stirring and dissolved. The finished solution is filtered through a 0.2 μm sterile filter and transferred into the containers provided for this purpose.

### EXAMPLE 2

| Oxymetazoline hydrochloride | 2.625 | g |
|---|---|---|
| Zinc gluconate | 200 | g |
| NaOH 1 mol/litre | 32 | ml |
| Sodium citrate | 65.94 | g |
| Water for injection purposes | 4699.435 | g |
| pH | 6.00 | |
| Osmolality | 280 | mOsm/l |

The aqueous solution is prepared analogously to Example 1.

### EXAMPLE 3

| Oxymetazoline hydrochloride | 2.625 | g |
|---|---|---|
| Zinc gluconate | 90.0 | g |
| Citric acid | 2.140 | g |
| Sodium citrate | 105.84 | g |
| Water for injection purposes | 4799.395 | g |
| pH | 6.00 | |
| Osmolality | 280 | mOsm/l |

The aqueous solution is prepared analogously to Example 1.

### EXAMPLE 4

### Comparative Example without Zinc Salt

| Oxymetazoline hydrochloride | 2.625 | g |
|---|---|---|
| Citric acid | 8.480 | g |
| Sodium citrate | 144.100 | g |
| Water for injection purposes | 4844.795 | g |
| pH | 5.99 | |
| Osmolality | 291 | mOsm/l |

The aqueous solution is prepared analogously to Example 1.

### EXAMPLE 5

| Xylometazoline hydrochloride | 1.000 | g |
|---|---|---|
| Zinc gluconate | 60.00 | g |
| NaOH, 1 mol/litre | 10.24 | ml |
| Sodium citrate | 8.288 | g |
| Water for injection purposes | 954.0 | g |
| pH | 6.0 | |
| Osmolality | 290 | mOsm/l |

The aqueous solution is prepared analogously to Example 1.

### EXAMPLE 6

| Oxymetazoline hydrochloride | 0.500 | g |
|---|---|---|
| Zinc gluconate | 60.00 | g |
| NaOH, 1 mol/litre | 22.0 | ml |
| Sodium citrate | 8.288 | g |
| Water for injection purposes | 942.1 | g |
| pH | 7.0 | |
| Osmolality | 302 | mOsm/l |

The aqueous solution is prepared analogously to Example 1.

### EXAMPLE 7

| | | |
|---|---|---|
| Oxymetazoline hydrochloride | 0.500 | g |
| Zinc gluconate | 60.00 | g |
| Citric acid | 1.441 | g |
| Sodium citrate | 8.288 | g |
| Water for injection purposes | 963.7 | g |

| pH | 5.0 | |
|---|---|---|
| Osmolality | 302 | mOsm/l |

The aqueous solution is prepared analogously to Example 1.

### EXAMPLE 8

| Oxymetazoline hydrochloride | 0.525 | g |
|---|---|---|
| Zinc gluconate | 60.00 | g |
| Sodium acetate | 9.935 | g |
| Water for injection purposes | 959.5 | g |
| pH | 6.0 | |
| Osmolality | 285 | mOsm/l |

The aqueous solution is prepared analogously to Example 1.

The stability of the formulation according to the invention was tested in a stress test. To this end, containers containing the solutions according to Example 1 and, for comparative purposes, containers containing solution according to Example 4 were stored at 30° C. and 65% relative atmospheric humidity (RH), RH and 40° C. and 75%. Before storage and after a storage time of 52 weeks or 26 weeks, 2 containers were removed both for the determination of the oxymetazoline content and also for the determination of the decomposition products thereof and investigated by means of high-pressure liquid chromatography (HPLC).

The HPLC chromatographic investigations was carried out with buffer solution pH 2.5/acetonitrile 828/172 (v/v) as eluent. Column: LiChrospher® 100 CN, detection at 215 nm. The results of the stability investigations are shown in Table 1.

TABLE 1
Stability data of oxymetazoline and zinc

| | | 0.05% of oxymatazoline | | 0.05% of oxymetazoline | | 0.05% of oxymetazoline | |
|---|---|---|---|---|---|---|---|
| | | without zinc | | with 1.8% of zinc | | with 6.0% of zinc | |
| | | (Example 4) | | (Example 3) | | (Example 1) | |
| Storage | Storage | Oxymetazoline | Hydrolysis | Oxymetazoline | Hydrolysis | Oxymetazoline | Hydrolysis |
| [weeks] | condition | [%] | product [%] | [%] | product [%] | [%] | product [% |
| 0 | | 101.7 | <0.05 | 100.6 | <0.05 | 102.5 | <0.05 |
| 26 | 30° C./65% RH | 103.4 | 0.33 | 101.9 | 0.27 | 104.0 | 0.27 |
| 26 | 40° C./75% RH | 100.2 | 1.50 | 99.6 | 1.17 | 98.3 | 0.98 |
| 52 | 30° C./65% RH | 101.5 | 0.68 | 101.1 | 0.58 | 102.5 | 0.59 |

The results clearly show that the formulation according to the invention has significantly increased stability compared with the comparative solution without zinc.

1. Aqueous pharmaceutical solution comprising at least oxymetazoline and/or xylometazoline, a zinc salt and a buffer salt.

2. Aqueous pharmaceutical solution according to claim 1, characterised in that the active compound present is/are oxymetazoline and/or xylometazoline in the form of its hydrochloride salt

3. Aqueous pharmaceutical solution according to claim 1, characterised in that the zinc salt present is zinc chloride, zinc lactate, zinc sulfate, zinc citrate, zinc acetate, zinc histidinate, zinc orotate, zinc aspartate and/or zinc gluconate

4. Aqueous pharmaceutical solution according to claim 3, characterised in that the zinc salt present is zinc gluconate

5. Aqueous pharmaceutical solution according to claim 1, characterised in that the buffer salt present is sodium citrate

6. Aqueous solution according to claim 1, characterised in that one or more acid(s) or one or more base(s) is (are) furthermore present

7. Aqueous solution according to claim 6, characterised in that the acid present is the respective acid forming the buffer salt, i.e. the acid form of the anion present in the buffer salt, or the base present is the base forming the buffer salt, i.e. the base form of the cation present in the buffer salt

8. Aqueous pharmaceutical solution according to claim 1, characterised in that the solution has a pH of pH 4 to pH 7.5

9. Aqueous pharmaceutical solution according to claim 8, characterised in that the solution has a pH of pH 5.0 to pH 7.2, in particular a pH of about pH 6.0

10. Aqueous pharmaceutical solution according to claim 1, characterised in that the solution has an osmolality of about 280 mOsm

11. Aqueous pharmaceutical solution according to claim 1, characterised in that oxymetazoline and/or xylometazoline is present in a concentration of 0.005% by weight to 1.0% by weight

12. Aqueous pharmaceutical solution according to claim 11, characterised in that the oxymetazoline and/or xylometazoline is present in a concentration of 0.01% by weight to 0.5% by weight, in particular in a concentration of 0.05% by weight to 0.1% by weight

13. Aqueous pharmaceutical solution according to claim 1, characterised in that the zinc salt is present in a concentration of 0.1% by weight to 10% by weight

14. Aqueous pharmaceutical solution according to claim 1, characterised in that the buffer salt is present in a concentration of 0.01% by weight to 3% by weight

15. Aqueous pharmaceutical solution according to claim 1, characterised in that this comprises about 0.005% by weight to 0.1% by weight of oxymetazoline and/or xylometazoline, 1% by weight to 10% by weight of zinc gluconate, and a citrate buffer having a pH of about 6.0

16. Aqueous pharmaceutical solution according to claim 1, characterised in that only one of the active compounds oxymetazoline and xylometazoline is present in the form of its hydrochloride salt

17. Use of the aqueous pharmaceutical solution according to claim 1 for local decongestion of the mucous membrane in the nose
